(19) United States
(12) Patent Application Publication
(10) Pub. No.: US 2004/0146461 A1
(43) Pub. Date: Jul. 29, 2004

(54) Oral contrast media composition for computerized axial tomographic examinations and method

(76) Inventors: Vincenzo Giuliano, Winter Springs, FL (US); Concetta Giuliano, Winter Springs, FL (US)

Correspondence Address: EDWARD M. LIVINGSTON, PA, 963 TRAIL TERRACE DRIVE, NAPLES, FL, 34103, US

(21) Appl. No.: 10/354,644

(22) Filed: Jan. 29, 2003

(51) Int. Cl. A61K 49/04

(52) U.S. Cl. 424/9.45

(57) ABSTRACT

A crystalline composition of pharmacologically acceptable non-toxic salt of diatrizoic acid and a low calorie non-sweetened drink mix provides an orally administrable gastrointestinal contrast medium which results in a sufficient and faster rate of contrast in poorly compliant patients undergoing computerized axial tomographic examinations for acute abdomen. The non-toxic salt of diatrizoic acid medium may consist of meglumine diatrizoate or sodium diatrizoate and the low calorie non-sweetened drink mix may be that sold with the trademark Crystal Light Methods of use include orally administering individual doses of approximately 8 ounces of the composition in beverage form a pre-determined period before the examination depending on the particular gastrointestinal area to be examined.

### BACKGROUND OF THE INVENTION

This invention relates to gastrointestinal contrast agents and more particularly to a composition providing gastrointestinal contract for computerized axial tomographic examinations of acute abdomen in a clinical setting in instances where a poorly compliant patient is limited by the amount of oral intake.

The clinical condition of acute abdomen often results in bowel distention and fluid-filled bowel loops which respond poorly to oral contrast media that contain salts of diatrizoic acid. There are commercially available salts of diatrizoic acid such as Gastrografin sold by Bracco Diagnostics, Inc. of Milan, Italy, and Gastroview sold by Mallinckrodt, Inc. of St. Louis, Mo. Both products contain identical amounts of pharmacologically acceptable non-toxic salts of diatrizoic acid comprising approximately 660 milligrams of meglumine diatrizoate and 100 milligrams of sodium diatrizoate solutions. The recommended dosage of these salts for computerized tomographic examinations is 25 milliliters (containing 9.17 grams of iodine) in 1000 milliliters of water, which is administered orally approximately 15 to 30 minutes prior to imaging. Unfortunately, individual dosing can be difficult to administer because of lack of a measuring tool. Furthermore, bowel opacification is often scant, dilute or unopacified due to a dilutional effect which occurs which this large volume of fluid mixed with the enteric fluid contained within distended bowels. As result, diarrhea is a common side effect due to the overload of a large amount of fluid volume in the gastrointestinal tract. Although in cases where the acute abdomen is complicated by a bowel perforation, diatrizoate salt solutions can permeate freely into the peritoneal cavity without adverse effects, unlike barium suspension, but it is colorless and therefore undetectable. Barium suspension induces peritonitis when free in the peritoneal cavity and is therefore contraindicated in the setting of an acute abdomen. Another problem can result from the fact that diatrizoate salts are also bitter and unpleasant tasting, thereby further contributing to reduced patient compliance.

Thus, a need exists for an oral composition of diatrizoate salts which provide a sufficient and faster rate of gastrointestinal contrast for computerized axial tomographic examinations in the clinical setting of acute abdomen, especially where bowel perforation is expected, which overcomes the above problems. The prior art contains contrast agents but none like the present invention, as follows:

| Patent Number | Inventor | Issue Date |
|---|---|---|
| 5,233,005 | Yudelson et al. | Aug. 10, 1993 |
| 4,735,795 | Robinson et al. | Apr. 5, 1988 |
| 5,360,604 | Ruddy et al. | Nov.1, 1994 |
| 6,414,857 | Henrichs et al. | Jul. 23, 2002 |
| 6,375,931 | Ostensen et al. | Apr. 23, 2002 |
| 6,409,671B1 | Eriksen et al. | Jun. 25, 2002 |
| 5,716,642 | Bagchi et al. | Feb. 10, 1998 |
| 4,474,747 | Dimo etal. | Oct. 2, 1984 |

### SUMMARY OF THE INVENTION

The primary object of the subject invention is to provide a composition which reduces the amount of oral intake required for sufficient gastrointestinal contrast.

Another object of the present invention is to provide a composition which reduces the incidence of diarrhea.

A further object of the present invention is to provide such a composition which can be provided in individual dosing thereby eliminating the need for measuring.

An even further object of the present invention is to provide a composition which is packaged for efficient, economical storage in bulk quantities.

An additional object of the present invention is to provide such a composition which is colored to aid in the identification of bowel perforation in an operative setting.

The present invention fulfills the above and other objects by providing an oral crystalline composition having a pharmacologically acceptable non-toxic salt of diatrizoic acid in a low-calorie, non-sweetened drink mix. The non-toxic salt of diatrizoic acid may be meglumine diatrizoate or sodium diatrizoate, the non-sweetened drink mix would preferably be the drink mix sold by Kraft General Foods under the trademark Crystal Light. Preferably the composition would contain 13.2 grams of meglumine diatrizoate, or 2.0 grams of sodium diatrizoate. The composition would be orally administered at a pre-determined time period prior to conducting the computerized axial tomographic examination, approximately 15 minutes in the case of the abdomen, 30 minutes in the case of the pelvic area and 45 to 60 minutes where visualization of the color or appendix is required.

The above and other objects, features and advantages of the present invention should become even more readily apparent to those skilled in the art upon a reading of the following detailed description of the preferred embodiments of the invention.

### DESCRIPTION OF THE PREFERRED EMBODIMENTS

Salts of diatrizoic acid are used as radiographic contrast materials suitable for intravascular injection and oral administration for visualization of internal body organs and structures. Pharmacologically acceptable and non-toxic salts of diatrizoic acid are referenced in the US Pharmacopeia and comprise meglumine diatrizoate and sodium diatrizoate. Meglumine diatrizoate is designated chemically as 1-deoxy-1-(methylamino )-D-glucitol 3,5-diacetamido-2,4,6-triodobenzoate. Sodium diatrizoate is designated chemically as monosodium 3,5-diacetamido-2,4,6-triiodobenzoate. The clinical pharmacology of diatrizoate salts for use as gastrointestinal contrast media is the high atomic weight of iodine, which produces adequate radiodensity for radiographic contrast of body tissues, and its poor absorption from the gastrointestinal tract. Sodium diatrizoate contains more iodine on a weight basis, and is therefore more effective as a radiographic contrast agent, but is limited in high doses by its toxicity. Meglumine diatrizoate contains less iodine, but its solutions tend to be more viscous and less toxic. Accordingly, combinations of meglumine diatrizoate and sodium diatrizoate are used in combination.

In practicing the invention, the mixture of dry ingredients, comprised of 13.2 grams of meglumine diatrizoate, 2.0 grams of sodium diatrizoate, and 1.8 grams of Crystal Light low calorie non-sweetened drink mix (sold by Kraft General Foods, Inc., Rye Brook, N.Y.) provides sufficient bowel contrast opacification, when dissolved in only 8 ounces (1 cup) of water, and prescribed as a prepared beverage. The beverage is orally administered 15 minutes prior to computerized axial tomographic imaging of the abdomen; 30 minutes for the pelvic area, for opacification of small bowel loops; and 45 to 60 minutes in cases where visualization of the colon or appendix is required. Improved patient compliance is encountered because of the pleasant taste. A lower incidence of diarrhea is encountered because of the relatively small amount of oral fluid intake, which also facilitates the dispersion of diatrizoate salts in distended, fluid-filled bowel loops. The inclusion of colored dyes contained in Crystal Light drink mix can aid in the visualization of free bowel perforations and collections to the naked eye, as encountered during surgical or interventional radiology procedures.

The composition further would preferably be divided into individual doses for easy administration. An individual dose would comprise approximately 8 ounces, or one cup, and would have 10 calories, 96 milligrams of sodium (4.2 milliequivalent), 7.3 grams of organically bound iodine, 0 sugars, and 0 protein and bear a warning that it contains phenylalanine.

The invention now having been fully described, it should be understood that it may be embodied in other specific forms or variations without departing from its spirit or essential characteristics. Accordingly, the embodiments described above are to be considered in all respects as illustrative and not restrictive. The scope of the invention being indicated by the appended claims rather than the foregoing description, and all changes which come within the meaning and range of equivalency of the claims to be embraced therein.

Having thus described our invention, we claim:

1. A composition for providing a gastrointestinal contrast medium in patients undergoing computerized axial tomographic examinations for acute abdomen, said composition comprising:
a non-toxic salt of diatrizoic acid; and
a liquid in which the non-toxic salt of diatrizoic acid is mixed.

2. The composition of claim 1 wherein:
the non-toxic salt of diatrizoic acid is meglumine diatrizoate.

3. The composition of claim 1 wherein:
said salt of diatrizoic acid is sodium diatrizoate.

4. The composition of claim 1 wherein:
the liquid is a low calorie, non-sweetened drink mix is from a group that includes Crystal Light drink mix.

5. The composition of claim 2 wherein:
the composition contains 13.2 grams of meglumine diatrizoate.

6. The composition of claim 3 wherein:
the composition contains 2.0 grams of sodium diatrizoate.

7. The composition of claim 4 wherein:
the composition contains 1.8 grams of Crystal Light drink mix.

8. The composition of claim 1 wherein:
the composition is contained in quantities of approximately 8 ounces for individual dosing.

9. A method for providing gastrointestinal contrast for computerized axial tomographic examinations for acute abdomen using a composition comprised of a non-toxic salt of diatrizoic acid and a liquid in which the non-toxic salt of diatrizoic acid is mixed, said method comprising:
orally administering the composition in the beverage form to a patient at a predetermined time period prior to said examination.

10. The method of claim 9 wherein:
the predetermined time period is approximately 15 minutes for imaging of the abdomen.

11. The method of claim 9 wherein:
the predetermined time period is approximately 30 minutes for imaging of the pelvic area.

12. The method of claim 9 wherein:
the predetermined time period is approximately 45 to 60 minutes for visualization of one from a group of organs consisting of the colon and appendix.

13. The method of claim 9 wherein:
the non-toxic salt of diatrizoic acid is meglumine diatrizoate.

14. The method of claim 9 wherein:
wherein the non-toxic salt of diatrizoic acid is sodium diatrizoate.

15. The method of claim 9 wherein:
the liquid is a low calorie, non-sweetened drink mix from a group that includes Crystal Light drink mix.

16. The method of claim 9 wherein:
the composition contains 13.2 grams of meglumine diatrizoate.

17. The method of claim 9 wherein:
the composition contains 2.0 grams of sodium diatrizoate.

18. The method of claim 9 wherein:
the composition contains 1.8 grams of Crystal Light drink mix.

19. The method of claim 9 wherein:
the composition is apportioned in quantities of approximately 8 ounces for individual dosing.
